(19) United States
(12) Patent Application Publication
(10) Pub. No.: US 2018/0104298 A1
(43) Pub. Date: Apr. 19, 2018

(54) ZANTHOXYLUM MYRIACANTHUM VAR. PUBESCENS ESSENTIAL OIL AND APPLICATION THEREOF IN PREPARATION OF DRUG FOR TREATING DIABETES

(71) Applicant: Affiliated Xishuangbanna Tropical Botanical Garden Chinese Academy of Science, Xishuangbanna (CN)

(72) Inventors: Ping ZHANG, Xishuangbanna (CN); Dahab MAHMOUD, Xishuangbanna (CN); YouKai XU, Xishuangbanna (CN); Huabin HU, Xishuangbanna (CN); Juan GUO, Xishuangbanna (CN)

(73) Assignee: Affiliated Xishuangbanna Tropical Botanical Garden Chines

(21) Appl. No.: 15/844,567

(22) Filed: Dec. 17, 2017

Related U.S. Application Data
(63) Continuation of application No. PCT/CN2016/108472, filed on Dec. 4, 2016.

(30) Foreign Application Priority Data
Dec. 7, 2015 (CN) 201510889596.X

(51) Int. Cl. A61K 36/758; A23L 33/115; A23L 33/00

(52) U.S. Cl. CPC A61K 36/758; A23L 33/115; A23V 2002/00; A61K 2236/33; A61K 2236/17; A23L 33/40

(57) ABSTRACT

The invention relates to a pneumatic brake release apparatus used on an elevator traction machine. By means of the technical solution, the air films in the air cabin are pushed by using compressed air to drive the push rod to transfer a thrust force, so that the locking arms are unfolded, and the locking brake and the brake release are implemented in a pneumatic way. Therefore, the problems in the prior art that a great amount of electric energy is consumed and the noise is difficult to eliminate can be solved, no noise is generated during operation, the safety is high, the structure is simple and the manufacturing cost is greatly reduced.

### CROSS-REFERENCE TO RELATED APPLICATIONS

This application is a continuation of International Patent Application No. PCT/CN2016/108472 with a filing date of Dec. 4, 2016, designating the United States, now pending, and further claims priority to Chinese Patent Application No. 201510889596.X with a filing date of Dec. 7, 2015. The content of the aforementioned applications, including any intervening amendments thereto, are incorporated herein by reference.

### TECHNICAL FIELD

The present invention belongs to the technical field of medicines, and relates to Zanthoxylum myriacanthum var. pubescens essential oil and an application thereof in preparation of a drug for treating diabetes.

### BACKGROUND OF THE PRESENT INVENTION

At present, antidiabetic drugs for treating diabetes have many side effects and cannot effectively control disease progression of diabetes. Most patients rapidly develop into a state which needs insulin injection. The fundamental reason is continuous deterioration of the pancreatic β cell function. Therefore, an anti-diabetic drug which is developed from traditional medicine with little side effects and capable of restoring β cell function will have a great potential.

A Dai medicine Zanthoxylum myriacanthum var. pubescens is extracted from the original plant Zanthoxylum myriacanthum var. pubescens (Huang) Huang, and belongs to rutaceae Zanthoxylum. The fruits, leaves and rhizomes of the Zanthoxylum myriacanthum var. pubescens can be used for making qi flow and dispelling cold, relieving abdominal distension, removing toxins and relieving swelling and pain and is mainly used for treating “Mianbaihe” (poisonous insect-bite), “Daxiehe” (centipede-bite), “Bingxiheng” (scabies) and “Bingdongfeinuanlong” (sore swollen furunculosis) In addition, due to a special lemon aroma, Zanthoxylum myriacanthum var. pubescens are often used for barbecuing, soup cooking, meat curing and other food processing procedures by minority nationality residents in Xishuangbanna District. At present, the prior art does not show that the Zanthoxylum myriacanthum var. pubescens has medicinal value in the treatment of diabetes.

### SUMMARY OF PRESENT INVENTION

With respect to the prior art, a purpose of the present invention is to provide Zanthoxylum myriacanthum var. pubescens essential oil or an extract containing the Zanthoxylum myriacanthum var. pubescens essential oil with a diabetes treatment activity as well as application of the Zanthoxylum myriacanthum var. pubescens essential oil and any extract containing the Zanthoxylum myriacanthum var. pubescens essential oil.

A technical solution for achieving the inventive purpose of the present invention is as follows:

In one aspect, the present invention provides the Zanthoxylum myriacanthum var. pubescens essential oil or an extract containing the Zanthoxylum myriacanthum var. pubescens essential oil. The Zanthoxylum myriacanthum var. pubescens essential oil is a volatile substance extracted from the Zanthoxylum myriacanthum var. pubescens plant.

Preferably, an extraction method of the Zanthoxylum myriacanthum var. pubescens essential oil in the present invention is selected from a steam distillation method, an extrusion method, a cold-maceration method or a solvent extraction method.

Preferably, Zanthoxylum myriacanthum var. pubescens essential oil in the present invention is prepared by the method as follows;

Adding the Zanthoxylum myriacanthum var. pubescens plant into water, soaking and/or performing ultrasonic treatment, heating to a boiling state, collecting distillate, extracting an organic phase of the distillate with an organic solvent, and volatilizing the organic phase to dryness, thereby obtaining the Zanthoxylum myriacanthum var. pubescens essential oil.

The Zanthoxylum myriacanthum var. pubescens plant in the present invention is selected from separate fruit, pericarp, seeds and stems of the Zanthoxylum myriacanthum var. pubescens or a combination of them.

Preferably, the Zanthoxylum myriacanthum var. pubescens plant is a coarse powder or fragments.

Further preferably, the organic solvent is ethyl ether.

A preparation method of the Zanthoxylum myriacanthum var. pubescens essential oil or an essential oil extract in the present invention specifically comprises the following steps: drying a fresh Zanthoxylum myriacanthum var. pubescens plant, and soaking the dried Zanthoxylum myriacanthum var. pubescens plant in warm water of 45-65° C. for 60-150 minutes, performing ultrasonic treatment with ultrasonic waves for 30-60 minutes, heating to a boiling state, extracting for 2-3 hours, extracting with ethyl ether, and volatilizing the ethyl ether to dryness, thereby obtaining the Zanthoxylum myriacanthum var. pubescens essential oil.

Through carefully designed and solid research, the present invention discovered that the Zanthoxylum myriacanthum var. pubescens essential oil or the extract containing the Zanthoxylum myriacanthum var. pubescens essential oil has effects of protecting the pancreas structure and protecting liver and kidney structures. In addition, in another aspect, the present invention provides a novel application of the Zanthoxylum myriacanthum var. pubescens, i.e. the present invention further provides applications of the Zanthoxylum myriacanthum var. pubescens, the Zanthoxylum myriacanthum var. pubescensessential oil or the extract containing the Zanthoxylum myriacanthum var. pubescens essential oil in preparation of a drug or functional food for treating diabetes, protecting pancreas structure, and protecting liver or kidney structure.

The present invention further provides a drug or functional food containing the Zanthoxylum myriacanthum var. pubescens, the above Zanthoxylum myriacanthum var. pubescens essential oil or the extract containing the Zanthoxylum myriacanthum var. pubescens essential oil.

According to our research, the present invention discovered that the Zanthoxylum myriacanthum var. pubescens essential oil in the present invention has little side effects and extraordinary curative effects, and the curative effects are based on restoring β cell morphology and functions, i.e., the Zanthoxylum myriacanthum var. pubescens essential oil has an effect of lowering blood glucose levels accompanied with restoration of the β cell morphology and functions. Such an action mechanism is completely different from a drug for simply inhibiting blood glucose increase and has excellent market development value.

Additional aspects, and advantages of the present invention may be partially given in description below. Partial additional aspects and advantages become apparent from the description below or learned through practice of the present invention.

### DESCRIPTION OF THE DRAWINGS

FIG. 1a shows an effect of Zanthoxylum myriacanthum var. pubescens essential oil of lowering blood glucose of a mouse;

FIG. 1b shows an effect of Zanthoxylum myriacanthum var. pubescens essential oil of restoring the insulin level of a mouse;

FIG. 2 shows an effect of Zanthoxylum myriacanthum var. pubescens essential oil of protecting pancreas, liver and kidney of a mouse;

FIG. 3 shows an effect of Zanthoxylum myriacanthum var. pubescens essential oil on insulin secretion in the pancreas islet of a mouse:

FIG. 4 shows an effect of Zanthoxylum myriacanthum var. pubescens essential oil on multiple antioxidant enzymes in liver and kidney of a mouse; and

FIG. 5 shows an effect of Zanthoxylum myriacanthum var. pubescens essential oil on creatinin and urea levels in urine.

### DETAILED DESCRIPTION OF PREFERRED EMBODIMENTS

The present invention is further described below in combination with drawings and embodiments.

### Embodiments

1. Material and Method

1.1 Preparation of Zanthoxylum myriacanthum var. pubescens essential oil: picking fresh Zanthoxylum myriacanthum var. pubescans fruits from Mengwang village, Xishuangbanna in November, and drying for later use; soaking the dried Zanthoxylum myriacanthurn var. pubescens fruits in warm water of 60° C. for 120 minutes; performing ultrasonic treatment for 50 minutes, heating to a boiling state, and extracting for 2.5 hours; and extracting with ethyl ether, and volatilizing the ethyl ether to dryness, thereby obtaining the Zanthoxylum myriacanthum var. pubescens essential oil.

1.2 Reagents and Instruments. STZ is Purchased from Sigma Company.

1.2.1 Establishment of an STZ-induced Diabetes Model

After one week acclimatization, 30 healthy mice are subjected to fasting for 12 hours, and 200 mg/kg of a prepared fresh STZ (Streptozotocin) solution is injected to an abdomen of each mouse. Oral gavage of a dextrose solution is immediately conducted for preventing hypoglycemia. Seven days later, blood is collected from tail veins and blood glucose is measured by using a glucometer (Bayer, Germany) after 12 hours from fasting of the mice, and model establishment is regarded to be successful if a blood glucose value is higher than or equal to 11.1 mmol/L. In addition 6 mice are taken as a normal control group.

1.2.2 Animal Grouping and Dosing Method

The method comprises steps: randomly dividing 12 diabetic mice subjected to successful model establishment into two groups, wherein each group comprises 6 mice, and the two groups respectively refer to a model group and a Zanthoxylum myriacanthum var. pubescens essential oil treatment group; the next day after grouping, continuously dosing the Zanthoxylum myriacanthum var. pubescens essential oil by oral gavage for a week; observing activity conditions of the mice in the whole test period, and monitoring weight, ingestion and water intake of the mice; collecting tail blood and measuring the blood glucose after food intake after detection is completed, collecting blood from veins around the eyes, separating plasma, measuring insulin (an insulin kit, Mercodia Company in Sweden), and fixing pancreas, liver and kidney tissues with 10% of formalin to prepare pathological sections.

1.2.3 Statistical Analysis of Data

All indexes of software experimental results are represented by mean±SEM. Differences of mean values of various groups are subjected to Anova multivariate statistical analysis by using Graphpad5. Turkey's test inspects that a statistical difference exists if p is more than 0.05.

2. Result

During the experimental period, mice in the normal group are neat in fur, glossy and normal in activities, food intake and water intake. The mice subjected to successful STZ model establishment are drooping in spirit, lusterless and slow in activities, increased in facilities and water intake, and increased in urine volume. The mice in the Zanthoxylum myriacanthum var. pubescens essential oil treatment group are decreased in food intake and water intake, decreased in urine volume and normal in activities.

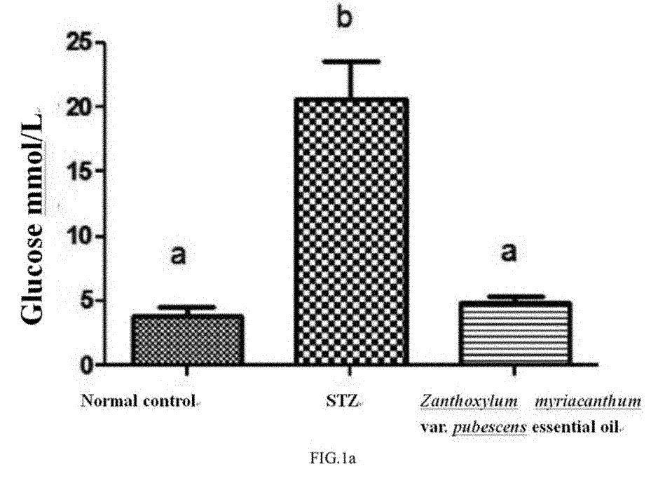

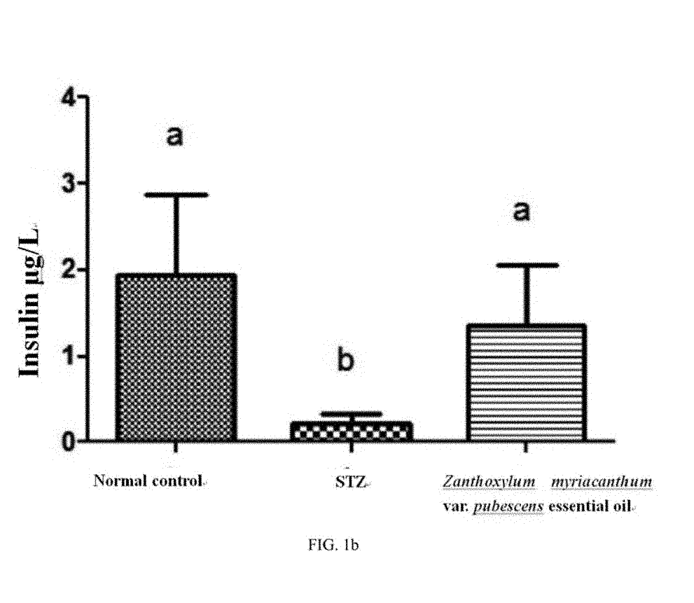

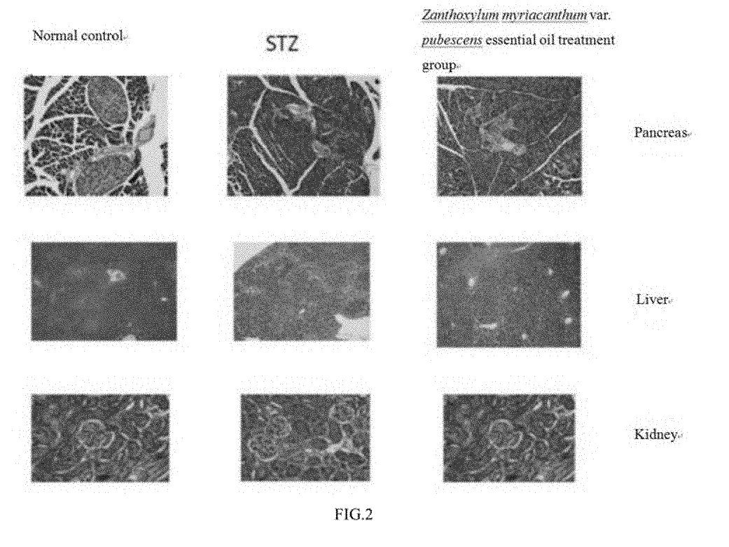

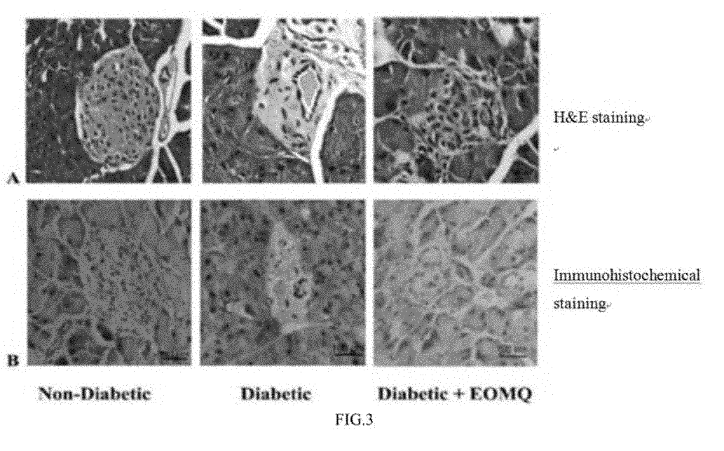

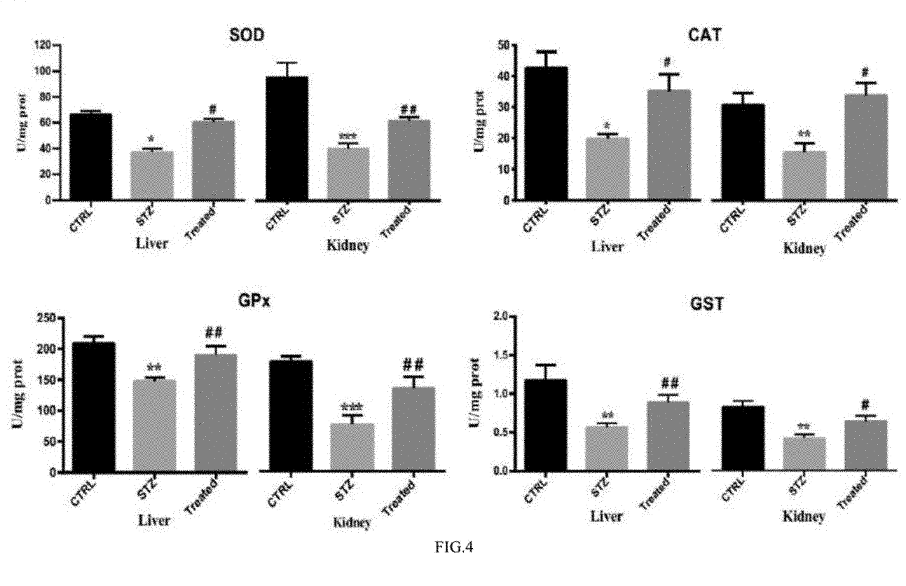

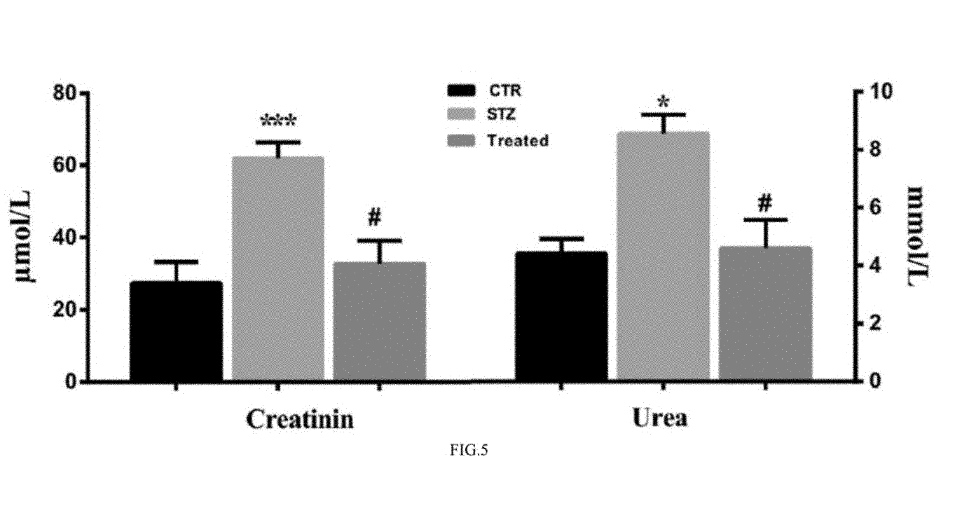

Through treatment within a week, a blood glucose level of the mice in the Zanthoxylum myriacanthum var. pubescens essential oil treatment group is reduced to a normal level, and the insulin level is restored to be normal (FIG. 1a, FIG. 1b and Table 1). For mice in the STZ model establishment group, pancreas of each mouse is obviously atrophied; islet cells secreting insulin are almost completely damaged; additionally, morphology of the liver is abnormal; obvious damage and lymphocytes infiltration exist around blood vessels; kidney tubules are slightly damaged; and glomerulus quantity is obviously decreased. For the mice in the Zanthoxylum myriacanthum var. pubescens essential oil treatment group, the pancreas structure is restored; the islet cells secreting insulin are restored; morphology of liver cells is close to be normal; necrocytosis around the blood vessels is obviously alleviated; obvious lymphocytes infiltration does not exist; the kidney structure is restored; the kidney tubules are not damaged; and the glomerulus quantity is restored (FIG. 2). Insulin antibody immunohistochemistry results show that insulin secreted in the pancreas islet is obviously increased after treatment with the Zanthoxylum myriacanthum var. pubescens essential oil (FIG. 3, wherein Diabetic EOMQ refers to the Zanthoxylum myriacanthum var. pubescens essential oil treatment group). Compared with the mice in the STZ diabetes group, levels of multiple antioxidant enzymes in livers and kidneys of the mice in the Zanthoxylum myriacanthum var. pubescens essential oil treatment group are restored to the normal level (FIG. 4, Table 2 and Table 3), and creatinin and urea levels in urine are also restored to be normal (FIG. 5 and Table 4). Therefore, a conclusion is drawn as follows: the Zanthoxylum myriacanthum var. pubescens essential oil has an effect of lowering the increased blood glucose levels of STZ-induced diabetic mice to the normal level and shows a protective effect on various tissues.

TABLE 1
Influences of zanthoxylum myriacanthum var. pubescens
essential oil on blood glucose and
insulin levels on STZ-induced diabetic mice

| | Blood glucose | |
|---|---|---|
| Group | (mmol/L) | Insulin (ug/L) |
| Normal control group | 3.83 ± 0.66 | 1.94 ± 0.94 |
| STZ | 20.57 ± 3.04 | 0.23 ± 0.11 |
| STZ + zanthoxylum myriacanthum var. | | |
| pubescens essential oil | 4.85 ± 0.45 | 1.36 ± 0.70 |

TABLE 2
Influences of zanthoxylum myriacanthum var. pubescens essential oil on
antioxidant enzymesof livers of STZ-induced diabetic mice

| | Liver (U/mg protein) | | | |
|---|---|---|---|---|
| Group | SOD | CAT | GPx | GST |
| Normal control group | 66.45 ± 1.40 | 42.68 ± 5.19 | 208.80 ± 11.51 | 1.18 ± 0.19 |
| STZ | 37.40 ± 2.41 | 19.78 ± 1.54 | 147.80 ± 5.89 | 0.57 ± 0.05 |
| STZ + zanthoxylum myriacanthum var. | 60.47 ± 2.51 | 35.25 ± 5.39 | 190.2 ± 6.33 | 0.89 ± 0.10 |
| pubescens essential oil | | | | |

TABLE 3
Influences of zanthoxylum myriacanthum var. pubescens essential oil on
antioxidant enzymes of kidneys of STZ-induced diabetic mice

| | Liver (U/mg protein) | | | |
|---|---|---|---|---|
| Group | SOD | CAT | GPx | GST |
| Normal control group | 95.96 ± 5.02 | 37.87 ± 4.81 | 176.40 ± 8.48 | 0.82 ± 0.08 |
| STZ | 40.07 ± 4.43 | 18.62 ± 4.49 | 75.15 ± 6.66 | 0.42 ± 0.05 |
| STZ + zanthoxylum myriacanthum var. | 61.74 ± 2.97 | 40.61 ± 4.96 | 133.2 ± 9.31 | 0.63 ± 0.07 |
| pubescens essential oil | | | | |

TABLE 4
Influences of zanthoxylum myriacanthum var. pubescens essential
oil on creatinin and urea in urine of STZ-induced diabetic mice

| | Creatinin | |
|---|---|---|
| Group | (umol/L) | Urea (mmol/L) |
| Normal control group | 27.46 ± 5.77 | 4.41 ± 0.51 |
| STZ | 61.95 ± 4.57 | 8.56 ± 0.66 |
| STZ + zanthoxylum myriacanthum var. | 32.78 ± 6.33 | 4.59 ± 0.98 |
| pubescens essential oil | | |

The mechanism of STZ-induced diabetes is to directly cause pancreas β cell death and drastic decrease in insulin secretion, leading tohyperglycemia, experimental diabetes, and a series of complications. Experimental results show that the Zanthoxylum myriacanthum var. pubescens essential oil can lower the blood glucose of the STZ-induced diabetic mice to the normal level. Specific components for achieving the blood glucose reducing effect in the Zanthoxylum myriacanthum var. pubescens essential oil need to be further studied, and mechanisms for promoting β cell function restoration and an effect for treating the complications of diabetes also need to be further studied. The effect of the Zanthoxylum myriacanthum var. pubescens essential oil for lowering the blood glucose of the STZ-induced diabetic mice showed in this study provides an experimental evidence for blood glucose lowering pharmacological effect of the Zanthoxylum myriacanthum var. pubescens, and provides an experimental basis for development of the Zanthoxylum myriacanthum var. pubescens as a drug for lowering blood glucose and controlling the complications of diabetes.

Although embodiments and comparison embodiments of the present invention are illustrated and described, those ordinary skilled in the art should be understood that these embodiments can be subjected to multiple changes, modifications, replacements and transformations on premise of not deviating from principles and tenets of the present invention. A scope of the present invention is defined by claims and equivalents thereof.

We claim:

1. A Zanthoxylum myriacanthum var. pubescens essential oil or an extract containing the Zanthoxylum myriacanthum var. pubescens essential oil, wherein the Zanthoxylum myriacanthum var. pubescens essential oil is a volatile substance extracted from the Zanthoxylum myriacanthum var. pubescens plant.

2. The Zanthoxylum myriacanthum var. pubescens essential oil or the essential oil extract according to claim 1, wherein an extraction method of the Zanthoxylum myriacanthum var. pubescens essential oil is selected from a steam distillation method, an extrusion method, a cold-maceration method or a solvent extraction method.

3. The Zanthoxylum myriacanthum var. pubescens essential oil or the essential oil extract according to claim 1, wherein the Zanthoxylum myriacanthum var. pubescens essential oil is prepared by the following method: adding a Zanthoxylum myriacanthum var. pubescens plant into water, soaking and/or performing ultrasonic treatment, heating to a boiling state, collecting distillate, extracting an organic phase of the distillate with an organic solvent, and volatilizing the organic phase to dryness, thereby obtaining the Zanthoxylum myriacanthum var. pubescens essential oil.

4. The Zanthoxylum myriacanthum var. pubescens essential oil or the essential oil extract according to claim 3, wherein the Zanthoxylum myriacanthum var. pubescens plant is selected from one or a combination of more of fruit, pericarp, seeds and stems of the Zanthoxylum myriacanthum var. pubescens.

5. The Zanthoxylum myriacanthum var. pubescens essential oil or the essential oil extract according to claim 3, wherein the Zanthoxylum myriacanthum var. pubescens plant is coarse powder or a fragment.

6. The Zanthoxylum myriacanthum var. pubescens essential oil or the essential oil extract according to claim 4, wherein the Zanthoxylum myriacanthum var. pubescens plant is a coarse powder or fragments.

7. The Zanthoxylum myriacanthum var. pubescens essential oil or the essential oil extract according to claim 3, wherein the organic solvent is ethyl ether.

8. The Zanthoxylum myriacanthum var. pubescens essential oil or the essential oil extract according to claim 3, wherein the method comprises the following steps: drying a fresh Zanthoxylum myriacanthum var. pubescens plant, and soaking the dried Zanthoxylum myriacanthum var. pubescens plant in warm water of 45-65° C. for 60-150 minutes; then performing ultrasonic treatment with ultrasonic waves for 30-60 minutes, heating to a boiling state, extracting for 2-3 hours, extracting with ethyl ether, and volatilizing the ethyl ether to dryness, thereby obtaining the Zanthoxylum myriacanthum var. pubescens essential oil.

9. An application of the Zanthoxylum myriacanthum var. pubescens and the Zanthoxylum myriacanthum var. pubescens essential oil or the extract containing the Zanthoxylum myriacanthum var. pubescens essential oil according to claim 1 in preparation of a drug or functional food for treating diabetes, protecting the pancreas structure, protecting the liver structure or protecting the kidney structure.

10. A drug or functional food containing the Zanthoxylum myriacanthum var. pubescens and the Zanthoxylum myriacanthum var. pubescens essential oil or the extract containing the Zanthoxylum myriacanthum var. pubescens essential oil according to claim 1.
